(19) United States
(12) Patent Application Publication
(10) Pub. No.: US 2003/0031728 A1
(43) Pub. Date: Feb. 13, 2003

(54) Antimicrobial polymeric tubing

(76) Inventors: Daniel H. Martin, Las Vegas, NV (US); Todd E. Davis, Las Vegas, NV (US); Dale R. Lewis, Medina, OH (US)

Correspondence Address: DANIEL MARTIN, Suite 14, 3155 E. Partick Lane, Las Vegas, NV, 89120, US

(21) Appl. No.: 09/924,511

(22) Filed: Aug. 9, 2001

(51) Int. Cl. A61K 33/38

(52) U.S. Cl. 424/618

(57) ABSTRACT

Antimicrobial polymeric tubing is disclosed comprising a polymer compounded with a silver ion-emitting additive. In a second embodiment, the tubing is coextruded, and the silver ion-emitting additive is compounded only with the polymer located on the interior of the tube.

### CROSS REFERENCE TO RELATED APPLICATIONS

Not Applicable

### STATEMENT REGARDING FEDERALLY SPONSORED RESEARCH OR DEVELOPMENT

Not Applicable

### BACKGROUND OF THE INVENTION

1. Field of the Invention

Polymeric tubings are commonly used in many applications including dental instrument tubing, catheters, soda fountains, drinking fountains, refrigerators, etc. Tubings typically need to be replaced or sanitized on a regular basis due to the layer of microbial growth known as biofilm that accumulates over time inside the tube. The current invention relates to improving the antimicrobial properties of polymeric tubing to prevent the layer of microbial growth from occuring.

2. Discussion of the Prior Art

There have been a number of efforts to produce polymeric tubing that exhibits antimicrobial properties. As shown in U.S. Pat. Nos. 4,723,950; 5,554,373; and U.S. patent application Ser. No. 09/777,121; organically bound arsenic, penicillin, tetracycline, triclosan, and other organic microbicides have been used to produce an antimicrobial effect within various polymeric tubes. The microbicide is mixed with the polymeric resin prior to extrusion, applied to the exterior of the extrusion, or used to impregnate the polymer after extrusion.

U.S. Pat. No. 5,709,672 utilizes gentian violet on the surface of catheters as a microbicide.

### BRIEF SUMMARY OF THE INVENTION

The present invention utilizes a silver ion-emitting additive such as silver zeolite, compounded with the polymer prior to extrusion, as a microbicide. Therefor, no microbicides are present on the interior surface of the tubing to leach into the fluid that flows within, and only nontoxic silver ions are used to inhibit microbial growth on the interior of the polymeric tubing and in the fluid.

### DETAILED DESCRIPTION OF THE INVENTION

The preferred embodiment of the present invention comprises silver zeolite or silver zinc zeolite, compounded with polyurethane or polyvinylchioride prior to extrusion of the tubing. The nature of the zeolite causes silver ions to be released on a continuous basis, both on the interior of the tube, and on the exterior. The silver ions kill any microbes they contact by blocking the cell respiration pathway of the microbe. Therefor, microbial growth is prevented on the interior and exterior of the tube, and microbes in the fluid that passes through the tube are destroyed.

An alternate embodiment comprises tubing made with two or more extruders. The first extruder provides a polymer compound to the exterior of the tube, and the second extruder provides a polymer compounded with a silver zeolite to the interior of the tube. This is useful when the antimicrobial property is not necessary or desired on the exterior of the tube, and decreases manufacturing cost due to less zeolite being required.

References herein to the details of the polymers and silver ion-producing additives are by way of example only and not intended to limit the scope of the claims which themselves recite those details regarded as important to the invention.

1. A tube comprising at least one polymer and at least one additive that sheds silver ions.

2. The tube of claim 1 wherein said additive comprises silver zeolite.

3. The tube of claim 1 wherein said additive comprises silver zinc zeolite.

4. The tube of claim 1 further defined as a catheter.

5. In combination:
a) a fluid dispensing apparatus, and
b) a tube comprising at least one silver-shedding additive.

6. The combination of claim 5 wherein said silver-shedding additive is further defined as silver zeolite.

7. The combination of claim 5 wherein said silver-shedding additive is further defined as silver zinc zeolite.

8. The combination of claim 5 wherein said fluid dispensing apparatus is defined as a dental syringe.

9. The combination of claim 5 wherein said fluid dispensing apparatus is defined as a dental handpiece.

10. The combination of claim 5 wherein said fluid dispensing apparatus is defined as a soda fountain.

11. The combination of claim 5 wherein said fluid dispensing apparatus is defined as a refrigerator.

12. In combination:
a) a fluid dispensing apparatus, and
b) a tube comprising:
1) an interior portion comprising a polymer compounded with at least one silver-shedding additive, and
2) an exterior portion comprising a polymer.

13. The combination of claim 12 wherein said interior portion and exterior portion are coextruded.

14. The combination of claim 12 wherein said silver-shedding additive comprises silver zeolite.

15. The combination of claim 12 wherein said silver-shedding additive comprises silver zinc zeolite.

16. The combination of claim 12 wherein said fluid dispensing apparatus is defined as a dental syringe.

17. The combination of claim 12 wherein said fluid dispensing apparatus is defined as a dental handpiece.

18. The combination of claim 12 wherein said fluid dispensing apparatus is defined as a soda fountain.

19. The combination of claim 12 wherein said fluid dispensing apparatus is defined as a drinking water dispenser.

20. The combination of claim 12 wherein said fluid dispensing apparatus is defined as a refrigerator.
